(19) United States
(12) Patent Application Publication
(10) Pub. No.: US 2012/0322061 A1
(43) Pub. Date: Dec. 20, 2012

(54) MACROMOLECULAR COMPOUND FOR DETECTING TARGET MATERIAL AND METHOD OF DETECTING TARGET MATERIAL BY USING THE SAME

(75) Inventors: Myoung-soon KIM, Seoul (KR); Jong-myeon PARK, Incheon (KR); Ye-ryoung YONG, Seoul (KR); Sea-hee KIM, Seoul (KR)

(73) Assignee: SAMSUNG ELECTRONICS CO., LTD, Suwon-si (KR)

(21) Appl. No.: 13/443,628

(22) Filed: Apr. 10, 2012

(30) Foreign Application Priority Data
Jun. 14, 2011 (KR) 10-2011-0057602

(51) Int. Cl. C12Q 1/68; C08F 222/36; G01N 33/53

(52) U.S. Cl. 435/6.11; 435/7.1; 526/238.2

(57) ABSTRACT

Macromolecular compound for detecting a target material in a biological sample, and a method of using same.

### CROSS-REFERENCE TO RELATED APPLICATIONS

This application claims the benefit of Korean Patent Application No. 10-2011-0057602, filed on Jun. 14, 2011, in the Korean Intellectual Property Office, the disclosure of which is incorporated herein in its entirety by reference.

### BACKGROUND

In-situ hybridization (ISH) is a technique for detecting a particular target nucleic acid directly from a cell or tissue fragment. As an initial ISH method, there is a fluorescence in-situ hybridization (FISH) technique. According to the FISH technique, to confirm the presence of a particular DNA sequence, without cell culture or DNA extraction, cells are fixed on a slide while the shape of a chromosome or nucleus is not changed and then are reacted with various probes in which a fluorescent material is attached to a DNA that has a complementary sequence to the particular sequence of a target nucleic acid to confirm the presence or location of the target nucleic acid, and mutation of a chromosome or gene is confirmed by using a fluorescent microscope. Because target nucleic acids are distinguishable from each other according to the fluorescent color of, for example, dye attached to probes, different particular nucleic acids are detectable in the same cell or tissue. However, due to the use of fluorescence, the detection test needs to be performed using the fluorescent microscope in a dark room to prevent exposure of a sample to light.

Despite the merit of the FISH technique that various target nucleic acids are detectable, an in-situ hybridization that uses an enzyme reaction has been introduced to overcome the inconvenience resulting from the observing in a dark room. This in-situ hybridization is referred to as a chromogenic in-situ hybridization (CISH). When the CISH technique is used, an optical microscope is used even in a general environment, not in a dark room in a laboratory for performing diagnosis, and the color of a sample may be maintained even when the sample is exposed to light. In the case of a silver in-situ hybridization (SISH) that uses an optical microscope like the CISH, a detection time is shorter than in conventional methods and enables automation. However, the CISH and SISH techniques may not be used to detect various target nucleic acids simultaneously. As far as known up to now, the CISH simultaneously detects three different nucleic acids and the SISH detects only one nucleic acid.

Accordingly, even with conventional techniques, there is a need to develop a compound that enables simultaneous detection of various different target materials.

### SUMMARY

Provided are macromolecular compounds for detecting a target material.

Also provided are methods of detecting a target material by using the macromolecular compounds.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

These and/or other aspects will become apparent and more readily appreciated from the following description of the embodiments, taken in conjunction with the accompanying drawings in which:

FIG. 1 is a diagram showing a process for synthesizing a water-soluble copolymer to which a dye and a probe are bonded; and

FIG. 2 provides a schematic depiction polymer beads to which probes and dye are bound as described herein.

### DETAILED DESCRIPTION

Reference will now be made in detail to embodiments, examples of which are illustrated in the accompanying drawings, wherein like reference numerals refer to like elements throughout. In this regard, the present embodiments may have different forms and should not be construed as being limited to the descriptions set forth herein. Accordingly, the embodiments are merely described below, by referring to the figures, to explain aspects of the present description.

An aspect of the present invention provides a macromolecular compound for detecting a target material, wherein the macromolecular compound includes a water-soluble copolymer including a first repeating unit to which a dye is bonded and a second repeating unit to which a probe is bonded

The macromolecular compound is used to detect a target material, such as a polynucleotide or a protein present in a biological sample, for example, a cell or a tissue, and may be used in an in-situ hybridization method by which the presence and/or location of the target material in a biological sample is visually identifiable, preferably with the naked eyes (i.e., with unaided visual inspection). Also, the macromolecular compound includes a dye with variable color according to an external stimulus. The inclusion of the dye enables simultaneous detection of two or more different target materials present in a biological sample.

According to an embodiment of the present invention, the first repeating unit and the second repeating unit may each be independently or commonly selected from the group consisting of acrylamide, poly-L-lysine, N-vinylpyrrolidone, N-acryloxysuccinimide, and pyrollo.

According to an embodiment of the present invention, the first repeating unit to which a dye is bonded and the second repeating unit to which a probe is bonded may be irregularly arranged at a molar ratio of between about 2:8 and about 8:2, for example, about 2:8, 3:7, 4:6, 5:5, 6:4, 7:3, or 8:2, and any intervening ratios. That is, the first repeating unit to which a dye is bonded is present at a level of about 20% to about 80% as compared to the second repeating unit to which a probe is bonded (which is present at a level of about 80% to about 20%). In other embodiments, the first repeating unit is present at level of about 30% to about 70%, or about 40% to about 60%, or even about 50% (i.e., approximately equal molar proportions of the first and second repeating units) Also the first repeating unit to which a dye is bonded and the second repeating unit to which a probe is bonded may each be polymerized in a line shape (linear configuration) or a branch shape (branched configuration).

According to an embodiment of the present invention, the macromolecular compound may further include a third repeating unit. Also, the third repeating unit may be identical to or different from the first repeating unit or the second repeating unit, and for example, may be selected from the group consisting of acrylamide, poly-L-lysine, N-vinylpyrrolidone, N-acryloxysuccinimide, and pyrollo. Due to the characteristics of the compounds that constitute the first to third repeating units, the resulting copolymer may be water-soluble.

According to an embodiment of the present invention, the third repeating unit may be irregularly arranged with respect to the first repeating unit to which a dye is bonded and the second repeating unit to which a probe is bonded at a molar ratio of between about 2:8 and about 8:2, for example, about 2:8, 3:7, 4:6, 5:5, 6:4, 7:3, or 8:2, and any intervening ratios. That is, the third repeating unit to which a dye is bonded is present at a level of about 20% to about 80% as compared to the first and second repeating units (which are collectively present at a level of about 80% to about 20%). In other embodiments, the third repeating unit is present at level of about 30% to about 70%, or about 40% to about 60%, or even about 50% (i.e., approximately equal molar proportions of the third repeating unit and the combined first and second repeating units)

According to an embodiment of the present invention, the molecular weight of the water-soluble copolymer may be in a range of about 1,000 to about 1,000,000, or about 5,000 to about 500,000, or about 10,000 to about 100,000.

According to an embodiment of the present invention, the dye may be selected from the group consisting of photochromic dye, electrochromic dye, thermochromic dye, and indicator dye. The photochromic dye may be selected from, for example, diarylethene-based derivatives, but is not limited thereto. The electrochromic dye may be, for example, ethyl viologen, but is not limited thereto. Also, the thermochromic dye may be, for example, selected from the group consisting of bianthrone, dixanthylene, and xanthylidenanthrone, but is not limited thereto. The indicator dye may be, for example, selected from the group consisting of phenol red, methyl red, methyl orange, bromophenolblue phenol phthalane, bromothymolblue, thymol blue, cresol red, crystal violet, and thymolphthaletin, but is not limited thereto.

According to an embodiment of the present invention, a target material may be included in a biological sample from which the target material is to be detected, and non-limiting examples of the target material are a polynucleotide, a protein, a lipid, and a carbohydrate.

Also, the probe may include two or more probes that specifically bond to different sites of the target material. The two or more probes may bond to two or more binding sites of one target material, or may bond to two or more different target materials present in a biological sample. According to an embodiment of the present invention, the probe may be a polynucleotide or an antibody. The polynucleotide may be usable when the target material is a polynucleotide or a protein, and may be a single-stranded or double-stranded DNA or RNA. If the target material is a polynucleotide, a polynucleotide that is used as the probe may be a single-stranded polynucleotide that substantially complementarily bonds to the polynucleotide as the target material.

Another aspect of the present invention provides a method of detecting a target material, wherein the method includes contacting a biological sample, which includes the target material, and a macromolecular compound as described herein; and confirming the presence and/or location of the target material in the biological sample in view of (by visual detection of) the color of the macromolecular compound comprising a dye that specifically binds to the target material.

The respective processes of the target material detection methods will now be described in detail.

First, the macromolecular compound is brought into contact with the biological sample including the target material.

According to an embodiment of the present invention, the biological sample may be a cell or a tissue. The contacting may also be referred to as hybridization and may be performed in an environment in which the target material specifically bonds to a probe included in the macromolecular compound. For example, if the target material and the probe are polynucleotides, the hybridization may be performed in a hybridization buffer solution (for example, including 50% formamide, 2×SSC, and 10% dextrane sulfate). Also, during the contacting, previous hybridization may be performed to increase accessibility with respect to the target material and to prevent non-specific bonds.

According to an embodiment of the present invention, the method may further include, after the contacting, changing the color of the macromolecular compound. According to an embodiment of the present invention, the color changing may be performed by, according to the kind of a dye bonded to the macromolecular compound, controlling a pH of a solution including the macromolecular compound, electrically stimulating the macromolecular compound, controlling the temperature of a solution including the macromolecular compound, or optically stimulating the macromolecular compound to change the color of the dye. Optical stimulation includes stimulation by light, such as visible light. By changing the color of the dye by using the methods described above, different target materials present at different sites of the biological sample may be detectable simultaneously.

Finally, the location of the target material in the biological sample is confirmed in view of color of the macromolecular compound specifically bonded to the target material.

After the macromolecular compound is bonded to the target material, remaining unbound macromolecules in the sample solution can be removed by any suitable technique, such as by separating the unbound macromolecules on the basis of size or molecular weight (e.g., by use of a membrane filter).

The dye bonded to the macromolecular compound according to an embodiment of the present invention is colored, and the color thereof changes due to an external stimulus, such as a pH, electricity, temperature, or light. Accordingly, by confirming the location of the macromolecular compound in the biological sample with the naked eyes, the location of the target material is easily identifiable.

One or more embodiments will now be described in further detail with reference to the following examples. These examples are for illustrative purpose only and are not intended to limit the scope of the one or more embodiments.

### Example 1

Example 1 illustrates the synthesis of polyacrylamide copolymer to which dye and oligonucleotide probe are bonded.

Polyacrylamide has high solubility, high chemical stability, and thermal stability, and shows relatively low adsorption with respect to biological macromolecules. Accordingly, the polyacrylamide is a compound that is suitable for an assay for in-situ hybridization.

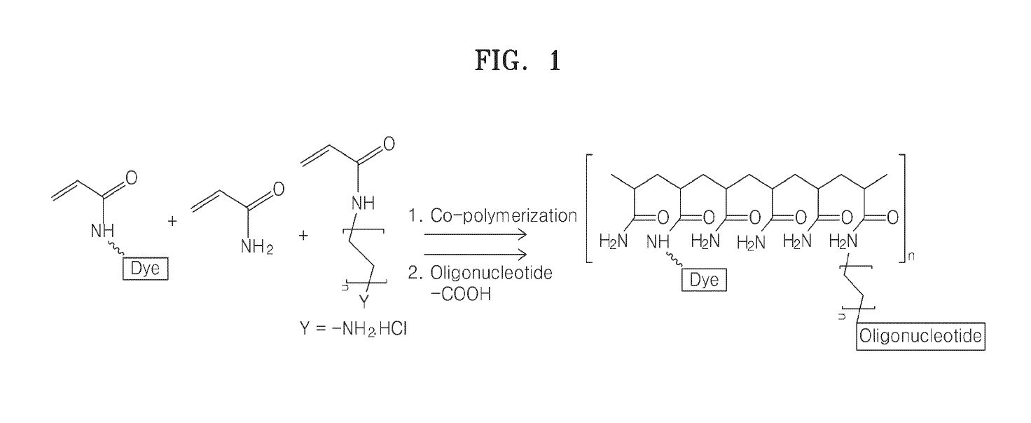

FIG. 1 shows a synthesis process of a polyacrylamide copolymer to which a dye and a probe are bonded. An acrylamide (5 mM) to which a dye was bonded, an acrylamide (0.5-1 mM) to which a probe was bonded, and a linking acrylamide (95 mM) were mixed and radical co-polymerization was performed thereon in 50% dimethyl formamide (DMF) containing 0.1% (w/v) ammonium sulfate and 0.5% (v/v) TEMED at a temperature of 25° C. for 16 hours, thereby obtaining a polyacrylamide copolymer bead to which the dye and the probe were bonded through their respective acrylamides and the linking acrylamide. In this case, the molecular weight of the polyacrylamide copolymer and the number of dyes and probes bonded to the polyacrylamide copolymer are controllable by adjusting the concentration of the acrylamide to which the dye was bonded, the acrylamide to which the probe was bonded, and the linking acrylamide, the molar ratio thereof, and the concentration of an initiator. Accordingly, the detection signal of a target material may be also amplifiable.

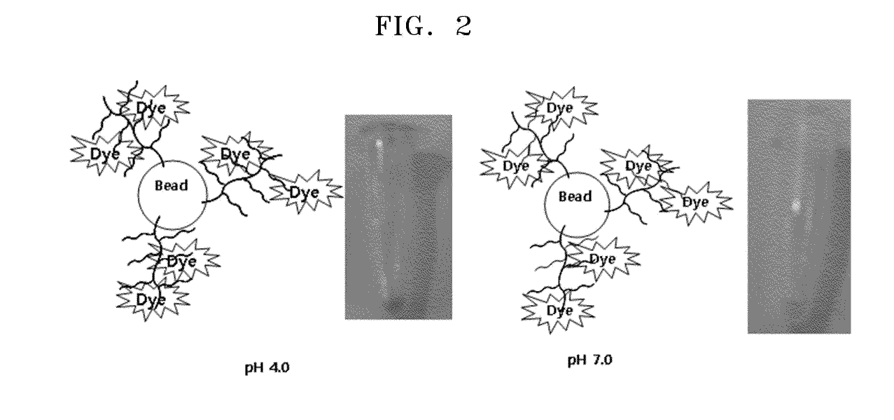

Methyl red, which is a pH indicator dye, can be used as the dye. When different probes are available for the same target material, more accurate detection results can be obtained. Also, the detection results are identifiable with the naked eye. The methyl red-bound acrylamide beads, prepared as described herein and schematically depicted in FIG. 2, visibly changed color according to the pH of the surrounding environment, appearing yellow in the presence of pH 4.0 and red in the presence of pH 7.0.

### EXAMPLE 2

Example 2 illustrates an in-situ hybridization test using polyacrylamide copolymer to which dye and oligonucleotide probe are bonded

An in-situ hybridization test is performed using the polyacrylamide copolymer to which the dye and probe were bonded and which was prepared according to Example 1. The polyacrylamide copolymer to which the dye and probe were bonded, prepared according to Example 1, are added to a cell or tissue to induce hybridization with DNA present in the cell or tissue and observed under a conventional optical microscope. In this case, various dyes and probes are allowed to bond to the copolymer to simultaneously detect different target materials located at different sites of a cell or tissue. In particular, like in Example 1, methyl red is used as a dye bonded to the copolymer to simultaneously detect different target materials in the same cell or tissue according to pH change.

When a macromolecular compound for detecting a target material and a method of detecting a target material by using the same according to the above embodiments of the present invention are used, the location of the target material in a biological sample is effectively detectable.

It should be understood that the exemplary embodiments described therein should be considered in a descriptive sense only and not for purposes of limitation. Descriptions of features or aspects within each embodiment should typically be considered as available for other similar features or aspects in other embodiments.

What is claimed is:

1. A macromolecular compound for detecting a target material, the macromolecular compound comprising a water-soluble copolymer comprising a first repeating unit to which a dye is bonded and a second repeating unit to which a probe is bonded.

2. The macromolecular compound of claim 1, wherein the first repeating unit and the second repeating unit are each independently acrylamide, poly-L-lysine, N-vinylpyrrolidone, N-acryloxysuccinimide, or pyrollo.

3. The macromolecular compound of claim 1, wherein the water-soluble copolymer comprises the first repeating unit to which the dye is bonded and the second repeating unit to which the probe at a molar ratio of between about 2:8 and about 8:2.

4. The macromolecular compound of claim 1, wherein the water-soluble copolymer of the macromolecular compound further comprises a third repeating unit.

5. The macromolecular compound of claim 4, wherein the third repeating unit is selected from the group consisting of acrylamide, poly-L-lysine, N-vinylpyrrolidone, N-acryloxysuccinimide, and pyrollo.

6. The macromolecular compound of claim 4, wherein the third repeating unit and the first and second repeating units to which the dye and probe are respectively bonded are irregularly arranged at a molar ratio of between 2:8 and 8:2.

7. The macromolecular compound of claim 1, wherein the water-soluble copolymer has a molecular weight of about 1,000 to about 1,000,000.

8. The macromolecular compound of claim 1, wherein the dye comprises at least one of a photochromic dye, an electrochromic dye, a thermochromic dye, or an indicator dye.

9. The macromolecular compound of claim 1, wherein the target material comprises a polynucleotide, protein, lipid, or carbohydrate.

10. The macromolecular compound of claim 1, wherein the macromolecular compound comprises two or more probes that specifically bond to different sites of the target material.

11. The macromolecular compound of claim 1, wherein the probe comprises a polynucleotide or antibody.

12. A method of detecting a target material, the method comprising:
contacting a biological sample, which comprises a target material, with a macromolecular compound comprising a water-soluble copolymer comprising a first repeating unit to which a dye is bonded and a second repeating unit to which a probe for the target material is bonded; and
identifying a location of the target material in the biological sample in view of color of the macromolecular compound that is specifically bound to the target material.

13. The method of claim 12, wherein the biological sample is a cell or tissue.

14. The method of claim 12, wherein the method further comprises, after contacting the biological sample with the macromolecular compound, changing color of the macromolecular compound.

15. The method of claim 14, wherein changing of the color of the macromolecular compound comprises changing the pH of a solution comprising the macromolecular compound, electrically stimulating the macromolecular compound, changing the temperature of a solution including the macromolecular compound, or optically stimulating the macromolecular compound.
